(19) United States
(12) Patent Application Publication
(10) Pub. No.: US 2013/0071444 A1
(43) Pub. Date: Mar. 21, 2013

(54) Amphiphilic Cationic Polymers and Methods of Use Thereof

(71) Applicants: Mingxing Wang, Matthews, NC (US); Qilong Lu, Charlotte, NC (US); Bo Wu, Matthews, NC (US); Peijuan Lu, Charlotte, NC (US)

(72) Inventors: Mingxing Wang, Matthews, NC (US); Qilong Lu, Charlotte, NC (US); Bo Wu, Matthews, NC (US); Peijuan Lu, Charlotte, NC (US)

(73) Assignee: The Charlotte-Mecklenburg Hospital Authority d/b/a Carolinas HealthCare System, Charlotte, NC (US)

(21) Appl. No.: 13/619,067

(22) Filed: Sep. 14, 2012

Related U.S. Application Data
(60) Provisional application No. 61/535,798, filed on Sep. 16, 2011.

(51) Int. Cl. A61K 47/34; A61K 48/00

(52) U.S. Cl. CPC A61K 47/34; A61K 48/00
USPC 424/400; 514/772.1; 514/44 A; 514/231.8; 435/375

(57) ABSTRACT

Amphiphilic cationic polymers comprising a biocompatible amphiphile linked to an organic cation are provided. The polymers complex with therapeutic agents and facilitate delivery of such therapeutic agents, particularly therapeutic nucleic acids, both in vitro and in vivo. Accordingly, the invention further provides methods of facilitating delivery of therapeutic and/or diagnostic agents to a cell and methods of treating a condition, such as a disease or infection, in an organism using the amphiphilic cationic polymers of the invention.

This application claims priority to U.S. Provisional Application No. 61/535,798, filed Sep. 16, 2011, the contents of which are incorporated herein in their entirety.

### FIELD OF THE INVENTION

The present invention relates to polymers comprising an amphiphilic backbone and an organic cation linked by means of a biodegradable linker. The polymers can be used to facilitate entry of therapeutic agents, including therapeutic nucleic acids, into cells. The polymers can also be used in methods of treating diseases, including muscular dystrophy.

### BACKGROUND OF THE INVENTION

The success of gene and oligonucleotide therapies relies upon the ability of systems to deliver the therapeutic genes and oligonucleotides to the target tissue efficiently and safely. Non-viral gene delivery systems, based on naked DNA/oligonucleotides, have advantages over viral vectors for simplicity of use and lack of specific immune response related to viral infection. However, naked DNA/oligonucleotides are difficult to be delivered into target cells in vivo. A number of synthetic gene delivery systems have been described to overcome the limitations of naked DNA/oligonucleotides, but their clinical relevance has been limited due to their low efficiency and high toxicity in vivo. For example, most of the non-viral vectors developed to date have been based on polycationic polymers, such as poly(L-lysine) (PLL), poly(L-arginine) (PLA), and polyethyleneimine (PEI). These polycationic polymers form interpolyelectronlyte complexes with negatively charged nucleic acids. The transfection efficiency of the cationic polymers is influenced by their molecular weight: polymers of high molecular weight (e.g., >20 KD) have better transfection efficiency than polymers of lower molecular weight. Unfortunately, cationic polymers with high molecular weight are also more cytotoxic (see US 2006/0093674 A1).

Several attempts have been made to circumvent the problems associated with conventional polycationic polymers and improve their transfection activity without increasing their cytotoxicity. For example, Lim et al. synthesized a degradable polymer, poly[α-(4-aminobutyl)-L-glycolic acid] (PAGA). See Pharm. Res., 17: 811-816 (2000). Other degradable polymers that have been synthesized and tested include poly-hydroxyproline ester (PHP ester) and networked poly(amino ester). See J. Am. Chem. Soc., 121:5633-5639 (1999); Macromolecules, 32:3658-3662 (1999); Bioconjugate Chem., 13:952-957 (2002). Although these alternative polymers condense DNA and transfect cells in vitro with low cytotoxicity, their overall low transfection activity and poor stability in aqueous solutions have limited their applicability.

Amphiphilic polymers, such as Pluronic™, poly(ethylene oxide)-block-poly(propylene oxide)-block-poly(ethylene oxide) (PEO-PPO-PEO triblock copolymer), are biocompatible and have been widely used as pharmaceutical adjuvants. Some of them have been approved by the FDA. Recently, Pluronic™ polymers such as F127 and SP1017 have been found effective in enhancing gene transfection efficiency of plasmid DNA in skeletal muscle. See, e.g., Lu et al., Gene Ther. 10:131-142 (2003); Lemieux et al., Gene Ther. 7:986-991 (2000); Pitard et al., Gene Ther. 13:1767-1775 (2002). In addition, Nguyen reported that a Pluronic™ P123-PEI 2 k conjugate mixed with free Pluronic™ P123 (1:9(w/w)) and DNA formed a stable and active formulation in vitro and in liver, and Vinogradov et al. reported that Pluronic™ P123-PEI 2 k mono-conjugates formulated with free Pluronic™ P123 increased transportation of phosphorothioate oligonucleotides across intestinal barrier as compared to PEI 25 k polymer. Nguyen et al., Gene Ther. 7:126-138 (2000). See also Cho et al., Macromolecular Research, 14: 348-353 (2006); Vinogradov et al., Journal of Drug Targeting, 12:517-526 (2004).

Despite progress in the field of non-viral gene/oligonucleotide delivery systems, there remains a need for improved compositions having greater transfection efficiency coupled with low toxicity.

### SUMMARY OF THE INVENTION

The present invention is based, in part, on the discovery that amphiphilic cationic polymers having intermediate size and hydrophilic-lipophilic balance (HLB) exhibit low cytotoxicity coupled with superior delivery of therapeutic agents, particularly nucleic acids, into cells.

Accordingly, in one aspect, the invention provides compositions comprising amphiphilic cationic polymers. In preferred embodiments, the amphiphilic cationic polymers have intermediate size and hydrophilic-lipophilic balance (HLB). In other preferred embodiments, the amphiphilic cationic polymer comprises a biocompatible amphiphile linked to an organic cation. The biocompatible amphiphile can be, for example, a poloxamer, a poloxamine, a polycaprolactone diol, a polycaprolactone polytetrahydrofuran block copolymer, a polysorbate polymer (e.g., a Tween series polymer), or a Triton polymer. The organic cation can be, for example, an amine, such as polyethylenimine (PEI), polypropylenimine (PPI), a low molecular weight amine, a dendrimer, or a polypeptide (e.g., poly-L-arginine or poly-L-lysine). In preferred embodiments, the linkage between the biocompatible amphiphile and the organic cation is provided by a biodegradable linker. In preferred embodiments, compositions of the invention further comprise a therapeutic or diagnostic agent. In certain embodiments, the therapeutic or diagnostic agent is a nucleic acid, such as an oligonucleotide or a transgene. In other embodiments, the therapeutic or diagnostic agent is a protein or a bulky, non-hydrophobic molecule. The therapeutic agent can be useful, for example, for treatment of a genetic disease, such as muscular dystrophy.

In another aspect, the invention provides pharmaceutical compositions comprising an amphiphilic cationic polymer of the invention in combination with a therapeutic or diagnostic agent. In certain embodiments, the pharmaceutical compositions further comprise a pharmaceutically acceptable carrier. In certain embodiments, the pharmaceutical composition is formulated for injection, such as intravenous, intramuscular, or intraperitoneal injection. In other embodiments, the pharmaceutical composition is formulated for oral delivery, nasal administration, or topical application.

In another aspect, the invention provides compositions for use in the manufacture of a medicament. In certain embodiments, the composition comprises an amphiphilic cationic polymer of the invention. In other embodiments, the composition comprises an amphiphilic cationic polymer of the invention in combination with a therapeutic or diagnostic agent. In certain embodiments, the medicament comprises a pharmaceutically acceptable carrier and is formulated for injection, such as intravenous, intramuscular, or intraperitoneal injection. In other embodiments, the medicament comprises a pharmaceutically acceptable carrier and is formulated for oral delivery, nasal administration, or topical application.

In another aspect, the invention provides methods of facilitating delivery of a therapeutic or diagnostic agent into a cell. The methods comprise contacting a cell with a composition comprising an amphiphilic cationic polymer of the invention in combination with a therapeutic or diagnostic agent. In certain embodiments, the methods comprise contacting the cell with a pharmaceutical composition comprising an amphiphilic cationic polymer of the invention in combination with a therapeutic or diagnostic agent and a pharmaceutically acceptable carrier. In certain embodiments, the cell is contacted in vitro, such as in a cell culture dish. In other embodiments, the cell is contacted in vivo. In certain embodiments, the contacting step comprises administering the composition to an organism comprising the cell such that the composition is able to contact the cell. In certain embodiments, the composition is administered to the organism by injection. In other embodiments, the composition is administered to the organism orally, nasally, or topically. In still other embodiments, the composition is administered to the organism by providing the organism with the composition in a formulation suitable for injection, oral ingestion, or nasal or topical application. In certain embodiments, the cell being contacted is from a primary culture of cells. In other embodiments, the cell being contacted is from an established cell line. In certain embodiments, the cell being contacted is selected from the group consisting of a muscle cell, a liver cell, an endothelial cell, a blood cell, an intestinal mucosal cell, a nasal mucosal cell, and a neuron. In preferred embodiments, the cell being contacted is a muscle cell.

In another aspect, the invention provides methods of treating a condition in an organism. The methods comprise administering to the organism a composition comprising an amphiphilic cationic polymer of the invention in combination with a therapeutic agent suitable for treating the organism's disease. In preferred embodiments, the therapeutic agent is a nucleic acid. In other embodiments, the therapeutic agent is a protein or a bulky, non-hydrophobic molecule. In certain embodiments, the organism being treated is an animal, such as a domesticated animal, a pet, a wild animal, a mammal or a bird. In preferred embodiments, the organism being treated is a mouse or a human. In preferred embodiments, the condition being treated is a genetic disease, such as muscular dystrophy. In other embodiments, the condition being treated is an infection, such as a bacterial, fungal, or viral infection.

Additional aspects and embodiments of the invention will be evident from the detailed description that follows.

### BRIEF DESCRIPTION OF THE DRAWINGS

FIG. 1 shows negative stain transmission electron microscopy (TEM) images of particles formed from PCM-05 polymer alone, PCM-05 complexed with DNA in a polymer:DNA ratio of 5:1 (w/w), DNA alone, and a polymeric mixture of Pluronic™ P85+PEI 1.2 k complexed with DNA in a polymer:DNA ratio of 5:1 (w/w).

FIG. 2 shows C2C12 cellular fluorescence 48 hours after treatment with PCM polymers of the invention complexed with 1 μg of a GFP transgene. PCM-04 (10 μg), PCM-05 (10 μg), PCM-07 (10 μg), PCM-08 (10 μg), and PCM-09 (5 μg) all induced transfection of the GFP transgene. C2C12 cells transfected with the GFP transgene using 2 μg of PEI 25 k are shown as a control.

FIG. 3 shows C2C12 GFP fluorescence 48 hours after treatment with 10 μg of a mixture of Pluronic L64+PEI 1.2 k, 10 μg of PCM-04, or 10 μg of PEI 1.2 k, each complexed with 1 μg of a GFP transgene.

FIG. 4 shows GFP fluorescence of CHO, C2C12, and H4IIE cells 48 hours after treatment with polymer PCM-04 complexed with a GFP transgene. The polymer:DNA ratio was 5:1 (w/w) for the CHO and C2C12 cells and 10:1 (w/w) for the H4IIE cells.

FIG. 5 shows exon skipping in C2C12 E50 cells after delivery of antisense oligonucleotides 2′-O-methyl phosphorothioate (2′-OMePS)-E50 (2 μg) or PMO-E50 (5 μg). Delivery of 2′-OMePS-E50 using polymer 021 (20 μg), 025 (100 μg), 044 (50 μg), or LF-2000 (4 μg) is shown in the top panel. Delivery of PMO-E50 using polymer 021 (50 μg), 025 (100 μg), 044 (100 μg), and Endo-porter (5 μg) is shown in the lower panel. The GFP fluorescence signal represents antisense oligonucleotide-mediated exon skipping, which restores the expression of a GFP transgene.

FIG. 6 shows delivery of PMO-E50 oligomer to C2C12 E50 cells grown in vitro, using dendron capped Tween-20 polymers (T20-Gn). The top series of images shows delivery using 0 μg, 5 μg, 10 μg, 20 μg, or 50 μg of T20-G2. The bottom series of images shows delivery using different generations (0, 1, 2, 3, 4, or 5) of T20-Gn polymers. The GFP fluorescence signal represents antisense oligonucleotide-mediated exon skipping, which restores the expression of a GFP transgene.

FIG. 7 shows the restoration of dystrophin in tibialis anterior (TA) muscles of mdx mice (age 4-6 weeks) two weeks after intramuscular (IM) injection of 2 μg antisense oligonucleotide PMO-E23 complexed with 5 μg of PCM-01 or PCM-05. Restoration of dytrophin following IM injection of 2 μg PMO-E23 alone is shown as a control. The expressed dystrophin appears as membrane (red) staining and the number of dystophin-positive fibers correlates with the efficiency of the PMO-E23 delivery.

FIG. 8 shows increased GFP expression in muscle cells in vivo following treatment with 10 μg of GFP expression vector alone or complexed with 10 μg of PCM-04, PCM-05, or PCM-08. 10 μg of GFP expression vector complexed with 2 μg of PEI 25 k is shown as a control. The GFP vector alone and complexes were locally injected into TA muscle of mdx mice. The treated muscles were dissected 5 days after the local injection and sections were cut from the muscles and viewed under fluorescence microscope.

### DETAILED DESCRIPTION OF THE INVENTION

In accordance with the present invention, compositions and methods for facilitating delivery of therapeutic and diagnostic agents into cells are provided. Compositions that find use in the methods of the invention comprise amphiphilic cationic polymers. Amphiphilic cationic polymers having intermediate size and hydrophilic-lipophilic balance (HLB) are particularly useful for practicing the methods of the invention as they have been found to exhibit low cytotoxicity while facilitating high levels of delivery of therapeutic agents, particularly nucleic acids, into cells.

The following definitions are provided to facilitate an understanding of the present invention:

As used herein, the term “polymer” denotes a molecule wherein at least a portion of the molecule is formed from the chemical union of two or more repeating units. The term “block copolymer” refers to conjugates of at least two different polymer segments, wherein each polymer segment comprises two or more adjacent units of the same kind.

As used herein, the term “hydrophobic” refers to the tendency of a molecule to partition into the non-polar, non-aqueous phase of a two phase system having a polar, aqueous phase and a non-polar, non-aqueous phase. The term “lipophilic” refers to the ability of a molecule to dissolve in a non-polar, non-aqueous liquid.

As used herein, the term “lipophobic” refers to the tendency of a molecule to partition into the polar, aqueous phase of a two phase system having a polar, aqueous phase and a non-polar, non-aqueous phase. The term “hydrophilic” refers to the ability of a molecule to dissolve in a polar, aqueous liquid.

As used herein, the term “amphiphilic” refers to a molecule that has both a hydrophobic portion and a lipophobic portion. Typically, in a two phase system having a polar, aqueous phase and a non-polar, non-aqueous phase, an amphiphilic molecule will partition to the interface of the two phases. The term “amphiphile” refers to an amphiphilic molecule.

As used herein, the term “organic cation” refers to a cationic molecule comprising carbon, hydrogen, and nitrogen atoms. Organic cations can further comprise other types of atoms, including oxygen atoms.

As used herein, the term “polycation” means a molecule having a plurality of positive charges distributed thereon. Polycations can be polymers. Examples of polycations include, without limitation, polyamines, such as spermine, polyspermine, spermidine, polyalkylenimines (e.g., polyethylenimine (PEI), polypropylenimine (PPI), etc.), and polyamidoamine (PAMAM).

As used herein, the term “biodegradable” refers to a molecule's ability to be broken down into less complex intermediates or end products by biological processes and/or biological agents (e.g., enzymes and other biological molecules having the ability to facilitate the breaking and transformation of chemical bonds). A “biodegradable linkage” is a chemical linkage between two different parts of a complex molecule, wherein the chemical linkage can be broken by biological processes and/or biological agents.

As used herein, a “substantially pure” molecule refers to a preparation comprising at least 50-60% by weight of the given molecule. More preferably, the preparation comprises at least 75%, 80%, or 85% by weight, and most preferably at least 90%, 95%, 98%, 99%, or more by weight of the given compound. Purity is measured by methods appropriate for the given compound (e.g., chromatographic methods, agarose or polyacrylamide gel electrophoresis, HPLC analysis, mass spectrometry, and the like).

As used herein, the terms “therapeutic agent,” “bioactive agent,” “drug” or any other similar term means any chemical or biological material or compound suitable for administration by the methods previously known in the art and/or by the methods taught in the present invention, which induces a desired biological or pharmacological effect. Such effects may include but are not limited to (1) having a prophylactic effect on an organism, such as preventing a condition, disease, or infection, (2) alleviating a condition, disease, or infection, or a symptom thereof, including, for example, alleviating pain or inflammation, and/or (3) completely eliminating a condition, disease, or infection from the organism. The effect may be local, such as providing for a local anesthetic effect, or it may be systemic.

The terms “therapeutic agent,” “bioactive agent,” and “drug” include broad classes of compounds normally delivered into the body, including, but not limited to: biomolecules, including nucleic acids, such as DNA, RNA, and oligonucleotides (e.g., siRNAs, oligonucleotide decoys, etc.), proteins, particularly pharmacologically active proteins, antibodies, vaccines, carbohydrates, and the like; and pharmaceutical compounds.

As used herein, the term “delivery” means transportation of an agent, such as a therapeutic agent, bioactive agent, drug, or diagnostic agent, into the cytoplasm and/or nucleus of a target cell or any other cell. Typically, the delivery process involves: (1) the agent coming into contact with a cell surface, either directly or indirectly by being complexed with another molecule which contacts the cell surface; (2) internalization of the agent by the cell, such as by endocytosis to an endosomal compartment; and (3) release of the agent into the cytoplasm of the cell. Delivery can be facilitated by improving the efficacy of at least one step in the delivery process such that there is an increase in the amount or percentage of the agent that reaches the cytoplasm and/or nucleus of the target cell. For example, a polymer can facilitate delivery of an agent by forming a complex with the agent, wherein the complex results in (1) an increase in the time duration or amount of cell surface contact experienced by the agent, (2) an increase in the amount or rate of internalization of the reagent by the cell, and/or (3) an increase in the amount or rate of release of the agent into the cytoplasm or nucleus of the cell.

As used herein, “transfecting” or “transfection” shall mean transport of nucleic acids from the environment external to a cell to the internal cellular environment, with particular reference to the cytoplasm and/or cell nucleus. Without being bound by any particular theory, it is to be understood that nucleic acids may be delivered into cells either after being encapsulated within or adhering to one or more amphiphilic cationic polymers of the invention, or being entrained therewith. Particular transfecting instances deliver a nucleic acid to a cell nucleus.

As used herein, “nucleic acid” and “nucleic acid molecule” are used interchangeably and refer to any DNA or RNA molecule, either single or double stranded. The nucleic acids can be genomic DNA, cDNA, short oligonucleotides, mRNA, tRNA, rRNA, siRNA, shRNA, hybrid sequences or synthetic or semi-synthetic sequences, of natural or artificial origin. Such nucleic acids can include one or more different types of modification. Accordingly, the nucleic acid can be variable in size, ranging from oligonucleotides to chromosomes, and may be of human, animal, vegetable, bacterial, viral, or synthetic origin. They may be obtained by any technique known to a person skilled in the art. The nucleic acids can be composed of standard bases (e.g., deoxy or dideoxy nucleotides) or modified bases (e.g., chemically modified bases). Modified bases can result, for example, in DNA or RNA molecules having a modified backbone structure (e.g., 2′-O-methyl oligonucleotides, peptide nucleic acids, etc.).

With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

As used herein, a “replicon” is any genetic element, such as a plasmid, cosmid, bacmid, phage or virus, which is capable of replication largely under its own control. A replicon may be either RNA or DNA and may be single or double stranded. A “vector” is a replicon, such as a plasmid, cosmid, bacmid, phage or virus, to which another genetic sequence or element (either DNA or RNA) may be attached so as to bring about the replication of the attached sequence or element. An “expression vector” refers to a vector which contains a sequence which can be transcribed into an RNA molecule, which in turn may be translated into a polypeptide or a protein, in a host cell or organism.

The term “gene” refers to a nucleic acid comprising an open reading frame encoding a polypeptide, including both exon and (optionally) intron sequences. The nucleic acid may also optionally include non-coding sequences such as promoter or enhancer sequences. The term “intron” refers to a DNA sequence present in a given gene that is not translated into protein and is generally found between exons.

The term “gene therapy” refers to the transfer of genetic material (e.g., DNA or RNA) of interest into a host organism (e.g., a human or other animal) to treat or prevent a condition, such as a genetic or acquired disease. The genetic material of interest may encode a product, such as a protein, of therapeutic value whose production in vivo is desired.

The term “ex vivo gene therapy” refers to the in vitro transfer of genetic material (e.g., DNA or RNA) of interest into a cell, which is then introduced (or reintroduced) into a host organism (see, for example, U.S. Pat. No. 5,399,346). The cells may be isolated from the host prior to transformation or may be obtained from a different source such as a different animal or human donor.

The phrase “small interfering RNA” or “siRNA” refers to a double stranded RNA molecule which inhibits the function or expression of a cognate mRNA (see, e.g. Ausubel et al., eds. Current Protocols in Molecular Biology, John Wiley and Sons, Inc., (1998)). A “short hairpin RNA” molecule or “shRNA” typically consists of short inverted repeats separated by a small loop sequence. Generally, one of the inverted repeats is complimentary to a gene target. The shRNA is typically processed into a siRNA within a cell by endonucleases. siRNAs and shRNAs specific for a protein of interest can downregulate its expression. (see, e.g., Myslinski et al. (2001) Nucl. Acids Res., 29:2502-09).

As used herein, “peptide” means peptides of any length, including full-length proteins. The terms “polypeptide” and “oligopeptide” are used herein without any particular intended size limitation, unless a particular size is otherwise stated. The only limitation to the peptide or protein drug which may be utilized is one of functionality.

As used herein, an “effective amount” means the amount of a therapeutic agent, bioactive agent, or drug that is sufficient to provide the desired local or systemic effect and performance at a reasonable risk/benefit ratio as would attend any medical treatment.

Amphiphilic Cationic Polymers

The invention provides amphiphilic cationic polymers that comprise a biocompatible amphiphile linked to an organic cation. Preferably, the linkage is provided by a biodegradable linker. In general, an amphiphilic cationic polymer of the invention will have a structure selected from the group consisting of:

OC-LN-H-L-LN-OC (i);

OC-LN-L-H-L-LN-OC (ii); and

OC-LN-H-L-H-LN-OC (iii),

wherein “H” is a hydrophilic segment, “L” is a lipophilic segment, “LN” is a biodegradable linker, “OC” is an organic cation, and the dashes are covalent chemical bonds, and wherein the hydrophilic and lipophilic segments together constitute a biocompatible amphiphile. Suitable hydrophilic segments include, for example, poly(ethylene oxide), polyglycerol (e.g., branched hydrophilic PG), branched aliphatic polyester (e.g., Bolton™ H2O), and the like. Suitable lipophilic segments include, for example, poly(propylene oxide) (PPO), polylactide (PLA), hydrocarbons (e.g., long-chain hydrocarbons, such as Capric acid, Undecylic acid, Lauric acid, Tridecylic acid, or Myristic acid, aromatic hydrocarbons, such as polyethylene glycol p-(1,1,3,3-tetramethylbutyl)-phenyl ether, and the like), cholesterol derivatives, and the like.

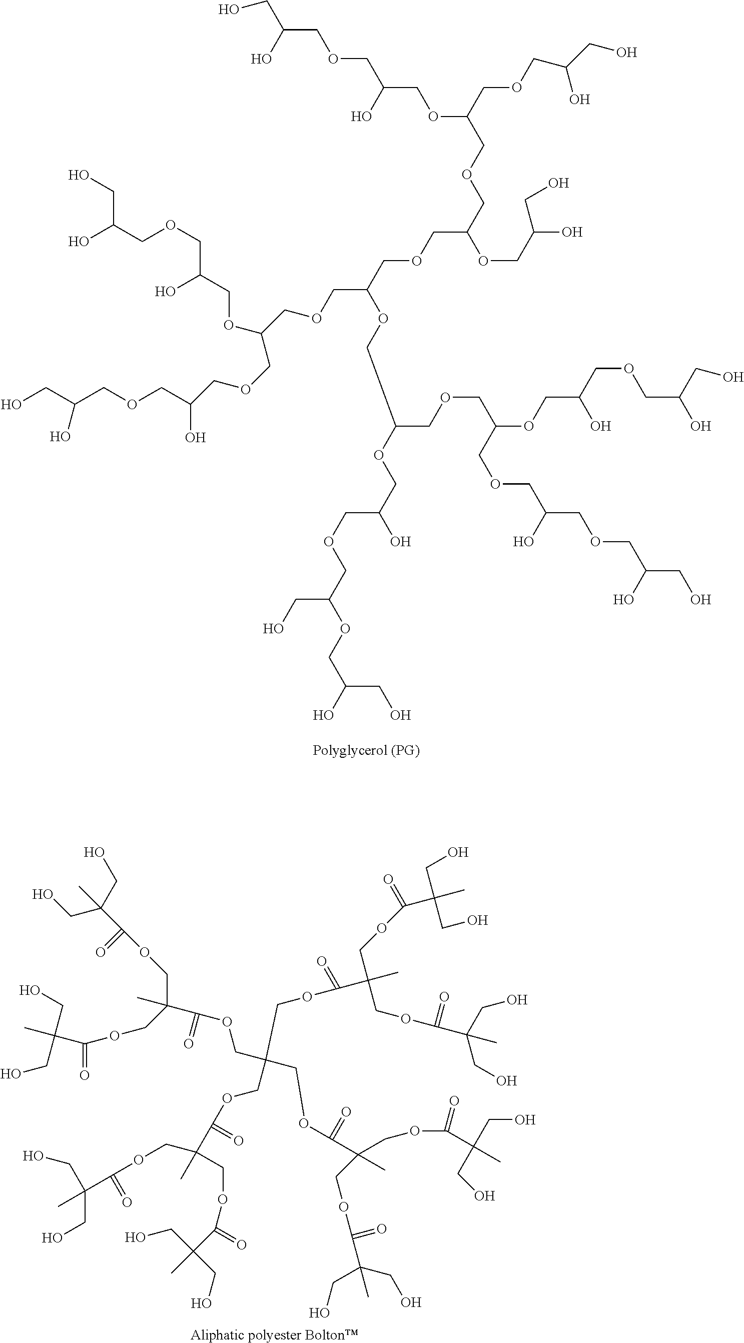 [SMILES: OCC(O)COCC(O)COCC(COCC(COCC(COCC(COCC(O)CO)OCC(O)COCC(O)CO)OCC(O)CO)OCC(COCC(COCC(O)COCC(O)CO)OCC(O)COCC(O)CO)OCC(O)COCC(CO)OCC(O)CO)OCC(O)COCC(O)CO ; CC(CO)(CO)C(=O)OCC(C)(COC(=O)C(C)(CO)CO)C(=O)OCC(COC(=O)C(C)(COC(=O)C(C)(CO)CO)COC(=O)C(C)(CO)CO)(COC(=O)C(C)(COC(=O)C(C)(CO)CO)COC(=O)C(C)(CO)CO)COC(=O)C(C)(COC(=O)C(C)(CO)CO)COC(=O)C(C)(CO)CO]

Preferably, the biocompatible amphiphile is a block copolymer selected from the group consisting of lipoloxamers, poloxamers (e.g., Pluronic® or Pluronic® R copolymers), poloxamines (e.g., Tetronic® or Tetronic® R copolymers), polylactide-poly(ethylene glycol) copolymers, polycaprolactone diol, polycaprolactone-polytetrahydrofuran copolymers, and the like. Alternatively, the biocompatible amphiphile can be a polysorbate polymer (e.g., from the Tween™ series, including Tween™-20, Tween™-40, Tween™-60, etc.) or a Triton™ polymer (e.g., Triton™ X-45, Triton™ X-100, Triton™ X-102, Triton™ X-114, Triton™ X-165, Triton™ X-305, etc.).

Lipoloxamers and poloxamers useful as biocompatible amphiphiles in an amphiphilic cationic polymer of the invention can have a formula selected from the group consisting of:

H[OCH₂CH₂]ₓ[OCH(CH₃)CH₂]_{y}OH (I);

H[OCH₂CH₂]ₓ[OCH(CH₃)CH₂]_{y}[OCH₂CH₂]_{z}OH (II); and

H[OCH(CH3)CH₂]ₓ[OCH₂CH₂]_{y}[OCH(CH₃)CH₂]_{z}OH (III)

wherein x, y, and z each have a value from about 5 to about 80. Preferably, x, y, and z each have a value from about 10 to about 65, about 15 to about 55, or about 20 to about 50. Persons skilled in the art will understand that formulas (I) through (III) are oversimplified in that, in practice, the orientation of the isopropylene radicals will be random.

Poloxamines useful as biocompatible amphiphiles in an amphiphilic cationic polymer of the invention can have a formula selected from the group consisting of (IV) or (V):

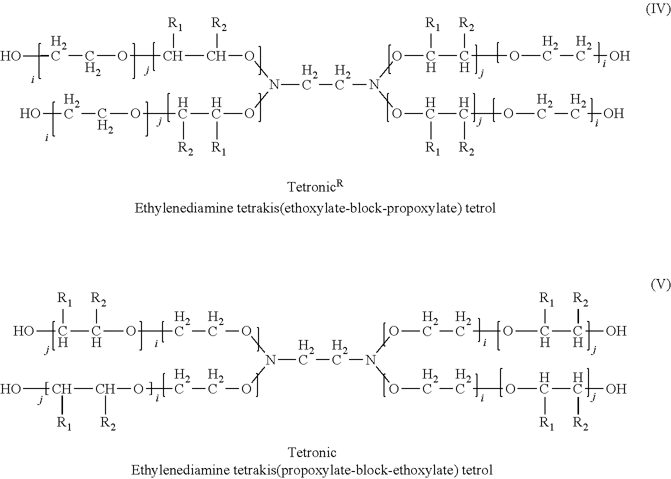

wherein i and j have values from about 2 to about 25, and wherein for each R₁, R₂ pair one is hydrogen and the other is a methyl group. Preferably, i and j each have a value from about 3 to about 20, or about 5 to about 15. Most preferably, i and j each have a value from about 6 to about 14, about 7 to about 13, or about 8 to about 12.

Preferably, the molecular weight of the polymers shown in formulas (I)-(V), above, is about 1000 Da to about 8000 Da, about 1900 Da to about 6500 Da, about 2400 Da to about 6000 Da, about 3000 Da to about 5500 Da, or about 3500 Da to about 5000 Da. Preferably, the molecular weight of the poly(ethylene oxide) of the polymer shown in formula (I)-(V), above, is about the same as the molecular weight of the poly(propylene oxide) in the polymer. For example, in preferred embodiments, the molecular weight of the poly(propylene oxide) in the polymer is about 35% to about 65%, about 40% to about 60%, about 45% to about 55%, or about 50% of the combined weight of the poly(propylene oxide) and poly(ethylene oxide) in the polymer.

Block copolymers comprising poly(ethylene oxide) and poly(propylene oxide) have been described, e.g., in U.S. Pat. No. 2,674,619 and by Santon, Am. Perfumer Cosmet. 72(4):54-58 (1958); Schmolka, Loc. cit. 82(7):25-30 (1967); and Schick (ed.), Non-ionic Surfactants, Dekker, N.Y., 1967 pp. 300-371. A wide variety of such polymers are commercially available (e.g., from BASF) and sold under such generic and trade names as lipoloxamers, poloxamers, Pluronic®, synperonics, meroxapol, Pluronic® R, poloxamines, or Tetronic®, or Tetronic® R. Commercially available poloxamer and meroxapol polymers preferred for use as a biocompatible amphiphile in an amphiphilic cationic polymer of the invention include, for example, Pluronic® L35, Pluronic® L44, Pluronic® L64, Pluronic® P65, Pluronic® P75, Pluronic® P84, Pluronic® P85, Pluronic® P104, Pluronic® P105, Pluronic® F127, Pluronic® R10R5, Pluronic® R17R4, Pluronic® R 17R8, Pluronic® R 22R4, Pluronic® R 25R4, Pluronic® R 25R5, and Pluronic® R 25R8.

Poly(ethylene oxide)-poly(propylene oxide) block copolymers can also be designed with hydrophilic blocks comprising a random mix of ethylene oxide and propylene oxide repeating units. To maintain the hydrophilic character of the block, ethylene oxide can predominate. Similarly, the hydrophobic block can be a mixture of ethylene oxide and propylene oxide repeating units. Such block copolymers are available from BASF under the tradename Pluradot™.

Additional biocompatible amphiphiles useful in the amphiphilic cationic polymers of the invention include block copolymer comprising polylactide or polycaprolactone and having a formula selected from the group consisting of:

H₃CO[CH₂CH₂O]ₓ[COCH(CH₃)O]_{y}H (VI);

HO[CH(CH₃)COO]ₓ[CH₂CH₂O]_{y}[COCH(CH₃)O]_{z}H (VII);

H[O(CH₂)₅CO]ₓCH₂CH₂OCH₂CH₂O[CO(CH₂)₅O]_{y}H (VIII); and

H[O(CH₂)₅CO]ₓ[CH₂CH₂O]_{y}[CO(CH₂)₅O]_{z}H (IX),

wherein the x in formula (VI) has a value of about 15 to about 30 and the y in formula (VI) has a value of about 2 to about 10, wherein the x and z in formula (VII) each have a value of about 10 to about 30 and the y in formula (VII) has a value of about 10 to about 250, wherein the x and y in formula (VIII) each have a value of about 2 to about 10, wherein the x and z in formula (IX) each have a value of about 2 to about 10 and the y in formula (IX) has a value of about 3 to about 40. In specific embodiments, the biocompatible amphiphile of formula (VI) can have an average value for x of about 22.5 and an average value for y of about 5. In other specific embodiments, the biocompatible amphiphile of formula (VII) can have average values for each of x and z of about 21 and an average value of y of about 20.5 or, alternatively, average values for each of x and z of about 14 and an average value for y of about 225. In other specific embodiments, the biocompatible amphiphile of formula (VIII) can have average values for each of x and y of about 2, about 4, or about 7.5. In still other specific embodiments, the biocompatible amphiphile of formula (IX) can have average values for each of x and z of about 4 and an average value for y of about 22.

Preferably, biocompatible amphiphiles used in an amphiphilic cationic polymer of the invention have an intermediate hydrophilic-lipophilic balance (HLB). The HLB value of a polymer reflects the balance of the size and strength of the hydrophilic groups and lipophilic groups present in the polymer. See, e.g., Attwood and Florence (1983), “Surfactant Systems: Their Chemistry, Pharmacy and Biology,” Chapman and Hall, New York. The HLB can be determined experimentally by, for example, the phenol titration method of Marszall (see, e.g., “Parfumerie, Kosmetik,” Vol. 60:444-48 (1979); Rompp, Chemistry Lexicon, 8^{th} Ed. (1983), p. 1750; and U.S. Pat. No. 4,795,643. Persons skilled in the art will understand that, as hydrophobicity increases, HBL decreases. In preferred embodiments, the biocompatible amphiphile used in an amphiphilic cationic polymer of the invention has an HLB of about 10 to about 26, about 10 to about 20, about 12 to about 19, about 14 to about 18, or most preferably about 15 to about 17.

Preferably, biocompatible amphiphiles used in an amphiphilic cationic polymer of the invention have an intermediate size and hydrophilic-lipophilic balance (HLB). For example, in certain embodiments, the biocompatible amphiphile has a size of about 1000 Da to about 10000 Da and an HLB of about 10 to about 26. In preferred embodiments, the biocompatible amphiphile has a size of about 1000 Da to about 8000 Da and an HLB of about 10 to about 20. In other preferred embodiments, the biocompatible amphiphile has a size of about 2000 to about 6000 and an HLB of about 14 to about 18. More preferably, the biocompatible amphiphile has a size of about 2500 to about 5000 and an HLB of about 15 to about 17. Commercially available polymers having intermediate size and HLB include, for example, Pluronic® L44, Pluronic® L64, Pluronic® P65, Pluronic® P75, Pluronic® P84, Pluronic® P85, and Pluronic® F127.

Organic cations suitable for use in the amphiphilic cationic polymers of the invention include, but are not limited to amines, including polyamines, such as linear or branched polyalkylenimines (e.g., polyethylenimine (PEI), polypropylenimine (PPI), etc.). Preferably, the organic cation is a low molecular weight polyalkylenimine. As used herein, a “low molecular weight polyalkylenimine” is a polyalkylenimine having a molecular weight of 3000 Da or less. For example, the low molecular weight polyalkylenimine can be branched polyethylenimine having a molecular weight between 200 and 3000, preferably 2000 Da or lower. Exemplary low molecular weight polyethylenimines include PEI-2 k (2000 Da), PEI-1.2 k (1200 Da), and PEI-0.8 k (800 Da). Alternatively, the low molecular weight polyalkylenimine can be branched polypropylenimine having a molecular weight between 200 and 3000, preferably 2000 Da or less.

Additional organic cations suitable for use in the amphiphilic cationic polymers of the invention include Jeffamines, dendrimers, and polypeptides (e.g., poly-L-arginine, poly-L-lysine, or a mixture of arginine and lysine). Suitable Jeffamines have the following structure:

H₃CO[CH₂CH(CH₃)O]ₓ[CH₂CH₂O]_{y}CH₂CH₂NH₂,

wherein x has an average value of about 2 to about 30, and wherein y has an average value of about 1 to about 35. Suitable dendrimers can be formed from diamines such as 1,2-ethanediamine, 1,3-propanediamine, 1,4-butanediamine, etc. Preferably, the Jeffamine, dendrimer, or polypeptide has a molecular weight of about 3000 Da or less. For example, preferred Jeffamines include M-600(XTJ-505) (PPO:PEO mol ratio 9:1; MW^{˜}600), M-1000(XTJ-506) (PPO:PEO mol ratio 3:19; MW^{˜}1000), M-2005 (PPO:PEO mol ratio 29:6; MW^{˜}2000), and M-2070 (PPO:PEO mol ratio 10:31; MW^{˜}2000); preferred dendrimers include polyethylenimine (PEI), polypropylenimine (PPI), and polypropylenimine diaminobutane (DAB) [DAB-dendr-(NH2)x] dendrimers; and preferred polypeptides include poly-L-lysine and poly-L-arginine, each having a molecular weight of about 500 Da to about 2000 Da.

Other organic cations suitable for use in the amphiphilic cationic polymers of the invention include low molecular weight amines. As used herein, a “low molecular weight amine” is an amine having a molecular weight of 500 Da or less. Preferably, the low molecular weight amine has a molecular weight of about 300 Da or less. The low molecular weight amine can be linear or cyclic and preferably includes two or more amines (e.g., two or more primary, secondary, or tertiary amines, or any combination thereof). Low molecular weight amines useful as organic cations include, but are not limited to, amines having one of the following structures:

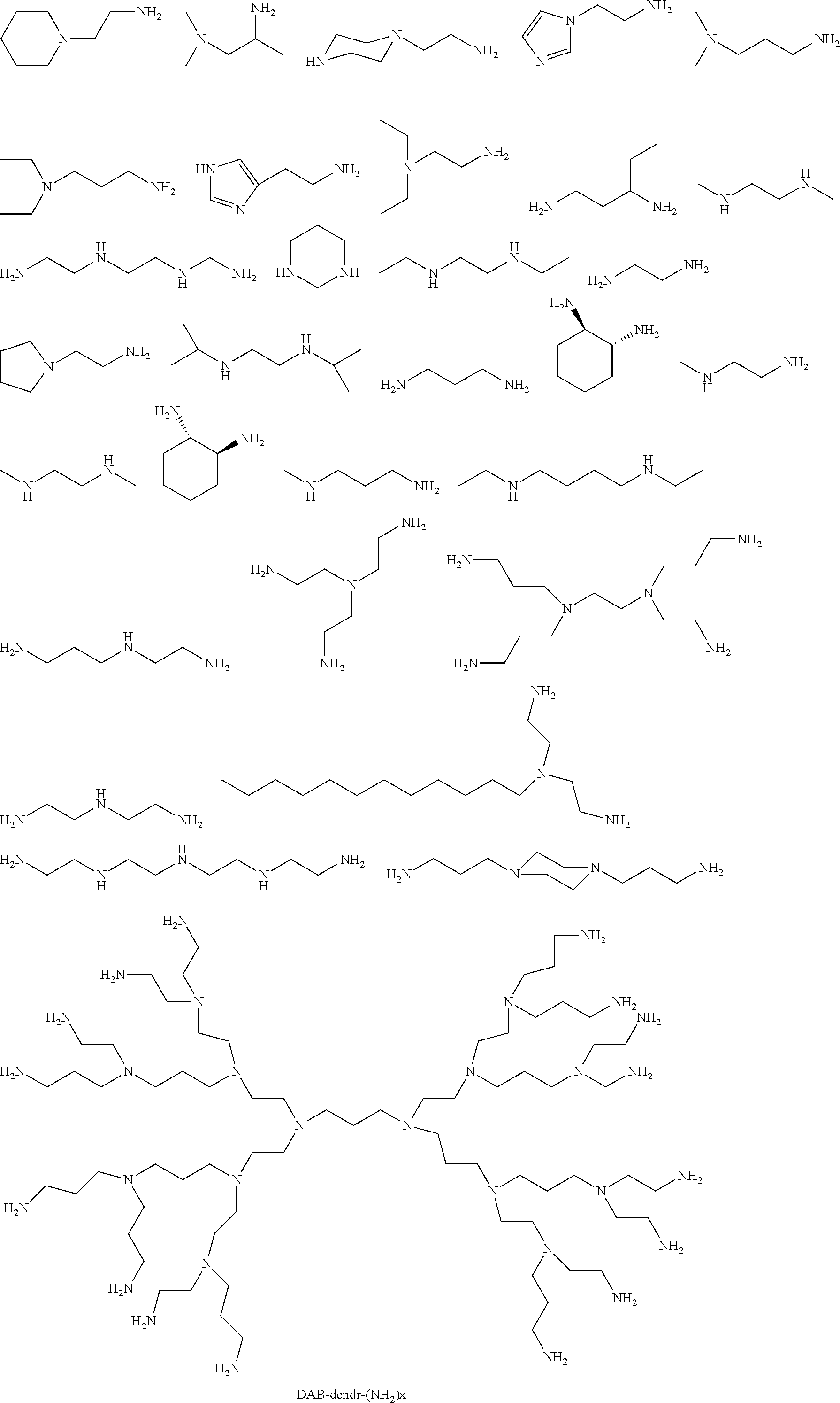

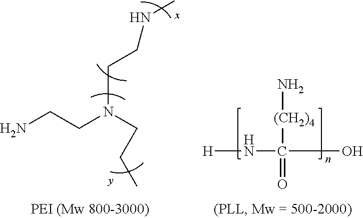

Other suitable low molecular weight amines will be obvious to persons skilled in the art.

Preferably, biocompatible amphiphiles and organic cations in the amphiphilic cationic polymers of the invention are linked together by a biodegradable linker. Suitable biodegradable linkers include, but are not limited to, amides, esters, urethanes, or di-thiols. In certain embodiments, the linker is simply a chemical bond, such as an ester amine or urethane bond. Persons skilled in the art can readily identify other suitable biodegradable linkers.

Accordingly, amphiphilic cationic polymers of the invention include, but are not limited to: PEI-2 k linked to Pluronic® P85, Pluronic® F127, Pluronic® L64, PEO-block-polylactide methyl ether (formula VI, above; average MW of polylactide about 350 Da; average MW of PEO about 1000 Da), polylactide-block-PEO-block-polylactide (formula VII, above; average MW of total polylactide about 3000 Da; average MW of PEO about 900 Da), polycaprolactone diol (formula VIII, above; average MW about 1250 Da), or polycaprolactone-block-polytetrahydrofuran-block-polycaprolactone (formula IX, above; average MW of total polycaprolactone about 1000 Da; average MW of polytetrahydrofuran about 1000 Da); PEI-1.2 k linked to Pluronic® P85, Pluronic® F127, Pluronic® L64, PEO-block-polylactide methyl ether (formula VI, above; average MW of polylactide about 350 Da; average MW of PEO about 1000 Da), polylactide-block-PEO-block-polylactide (formula VII, above; average MW of total polylactide about 3000 Da; average MW of PEO about 900 Da), polycaprolactone diol (formula VIII, above; average MW about 1250 Da), or polycaprolactone-block-polytetrahydrofuran-block-polycaprolactone (formula IX, above; average MW of total polycaprolactone about 1000 Da; average MW of polytetrahydrofuran about 1000 Da); PEI-0.8 k linked to Pluronic® P85, Pluronic® F127, Pluronic® L64, PEO-block-polylactide methyl ether (formula VI, above; average MW of polylactide about 350 Da; average MW of PEO about 1000 Da), polylactide-block-PEO-block-polylactide (formula VII, above; average MW of total polylactide about 3000 Da; average MW of PEO about 900 Da), polycaprolactone diol (formula VIII, above; average MW about 1250 Da), or polycaprolactone-block-polytetrahydrofuran-block-polycaprolactone (formula IX, above; average MW of total polycaprolactone about 1000 Da; average MW of polytetrahydrofuran about 1000 Da); bis-aminopropyl piperazine (BAPP) linked to Pluronic® P85, Pluronic® F127, Pluronic® L64, Tween-20, PEO-block-polylactide methyl ether (formula VI, above; average MW of polylactide about 350 Da; average MW of PEO about 1000 Da), polylactide-block-PEO-block-polylactide (formula VII, above; average MW of total polylactide about 3000 Da; average MW of PEO about 900 Da), polycaprolactone diol (formula VIII, above; average MW about 1250 Da), polycaprolactone-block-polytetrahydrofuran-block-polycaprolactone (formula IX, above; average MW of total polycaprolactone about 1000 Da; average MW of polytetrahydrofuran about 1000 Da), polyglycerol (PG), or aliphatic polyster Bolton (such as H20); poly-L-lysine (MW about 1250 Da) linked to Pluronic® P85, Pluronic® F127, Pluronic® L64, PEO-block-polylactide methyl ether (formula VI, above; average MW of polylactide about 350 Da; average MW of PEO about 1000 Da), polylactide-block-PEO-block-polylactide (formula VII, above; average MW of total polylactide about 3000 Da; average MW of PEO about 900 Da), polycaprolactone diol (formula VIII, above; average MW about 1250 Da), or polycaprolactone-block-polytetrahydrofuran-block-polycaprolactone (formula IX, above; average MW of total polycaprolactone about 1000 Da; average MW of polytetrahydrofuran about 1000 Da); and arginine linked to Pluronic® P85, Pluronic® F127, Pluronic® L64, PEO-block-polylactide methyl ether (formula VI, above; average MW of polylactide about 350 Da; average MW of PEO about 1000 Da), polylactide-block-PEO-block-polylactide (formula VII, above; average MW of total polylactide about 3000 Da; average MW of PEO about 900 Da), polycaprolactone diol (formula VIII, above; average MW about 1250 Da), or polycaprolactone-block-polytetrahydrofuran-block-polycaprolactone (formula IX, above; average MW of total polycaprolactone about 1000 Da; average MW of polytetrahydrofuran about 1000 Da); and Tween series (average MW about 3000 Da or less).

Amphiphilic cationic polymers of the invention can be synthesized starting with commercially available biocompatible amphiphiles (e.g., Pluronic polymers) and organic cations (e.g., PEI, poly-L-lysine, arginine), using synthetic methods well-known in the art to link the biocompatible amphiphiles to the organic cations, preferably using biodegradable linkers. For example, the following synthetic approach (Scheme-1) can be used to synthesize amphiphilic cationic polymers of the invention having ester-amine linkages.

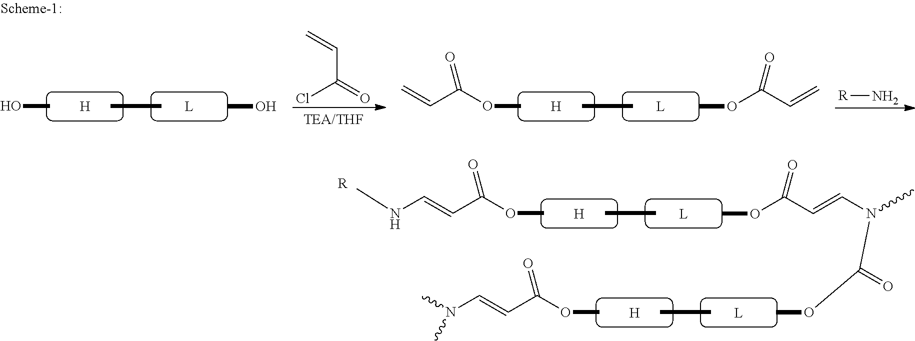

In Scheme-1, R represents an organic cation.

The following synthetic approach (Scheme-2) can be used to synthesize amphiphilic cationic polymers of the invention having urethane linkages.

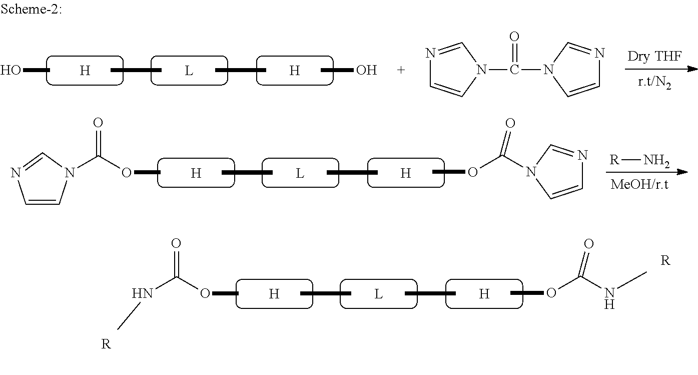

In Scheme-2, R represents an organic cation.

Alternatively, amphiphilic cationic polymers of the invention having urethane linkages and comprising cationic dendron-capped amphiphiles can be synthesized according to the following synthetic approach (Scheme-3).

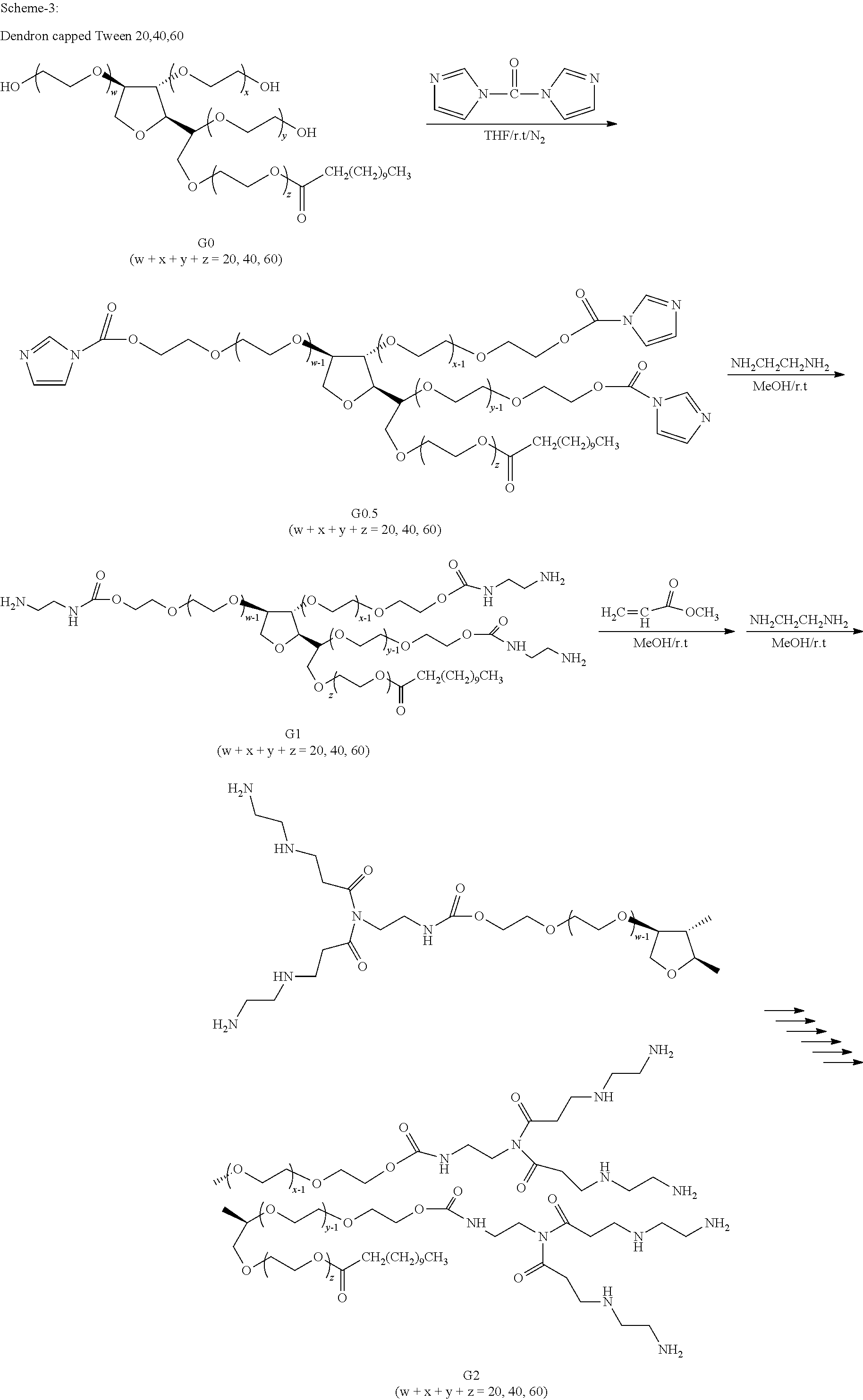

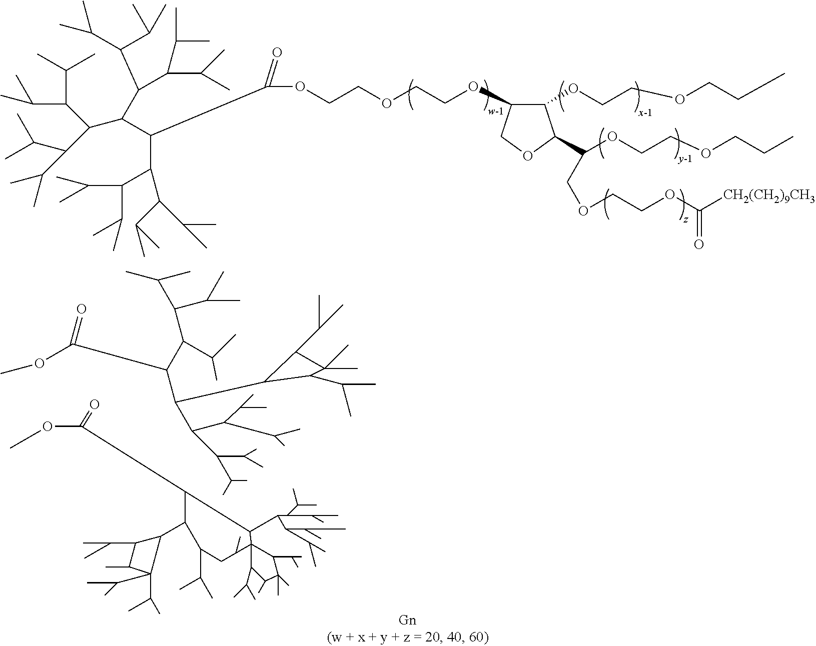

Amphiphilic cationic polymers of the invention having urethane linkages and comprising amino acid/polypeptide-modified amphiphiles (e.g., arginine-modified Pluronic® polymers) can be synthesized according to the following synthetic approach (Scheme-4).

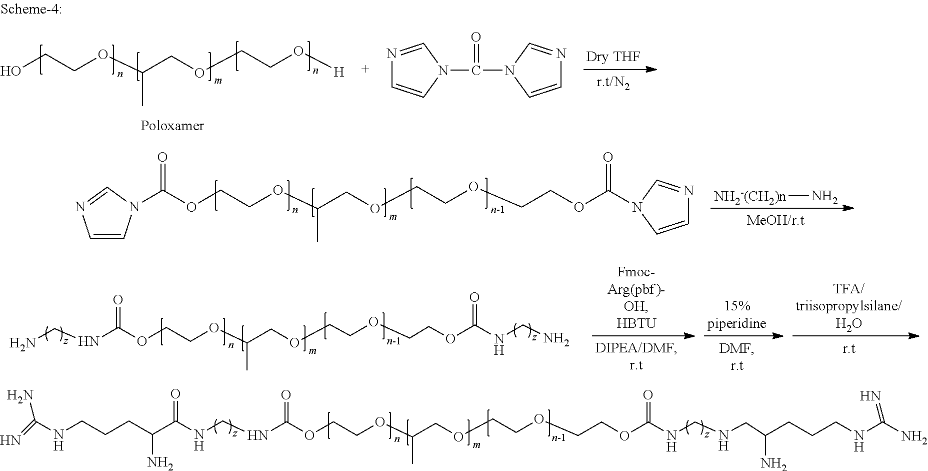

Persons skilled in the art will understand that similar synthetic approaches can be used to synthesize many different amphiphilic cationic polymers of the invention. Moreover, biocompatible amphiphiles and organic cations not available commercially can be readily synthesized using standard synthetic approaches well-known in the art.

Compositions

The invention also provides compositions comprising one or more (e.g., a mixture of) amphiphilic cationic polymers of the invention (e.g., one or more substantially pure amphiphilic cationic polymer). In certain embodiments, compositions of the invention consist essentially of one or more amphiphilic cationic polymers (e.g., one or more substantially pure amphiphilic cationic polymers). In other embodiments, compositions of the invention consist of one or more amphiphilic cationic polymers (e.g., one or more substantially pure amphiphilic cationic polymers). In preferred embodiments, compositions of the invention comprise one or more amphiphilic cationic polymers (e.g., one or more substantially pure amphiphilic cationic polymers) in combination with a therapeutic agent and/or a diagnostic agent. Compositions comprising one or more amphiphilic cationic polymers in combination with a therapeutic and/or diagnostic agent (i.e., pharmaceutical compositions) can further comprise a pharmaceutically acceptable carrier. Pharmaceutical compositions can be formulated for administration by a particular route (e.g., intravenous, intramuscular, or intraperitoneal injection; oral delivery; nasal administration; or topical application). Suitable methods for formulating pharmaceutical compositions comprising polymers, such as amphiphilic cationic polymers of the invention, are well-known in the art.

Therapeutic agents suitable for inclusion in the compositions of the invention include nucleic acids, proteins, and other chemical compounds (e.g., pharmaceutical drugs). In certain preferred embodiments, the therapeutic agent is a nucleic acid. The nucleic acid can be DNA, RNA, or a modified nucleic acid, such as a peptide nucleic acid (PNA) or a nucleic acid comprising 2′-O-methyl nucleotides. The nucleic acid can comprise an entire gene or cDNA, or a fragment thereof, such as a promoter fragment (e.g., an oligonucleotide decoy sequence comprising one or more transcription factor binding sites and/or an enhancer sequence), an intron sequence, an intron-exon junction sequence, a coding sequence, an antisense sequence, etc. The nucleic acid can be single or double stranded. Certain preferred nucleic acids include an open reading frame encoding a functional protein. Other preferred nucleic acids include antisense oligonucleotides or siRNAs that induces gene silencing or exon skipping. Still other preferred nucleic acids include a double-stranded oligonucleotide decoy sequence capable of influencing the transcription of a target gene. Use of nucleic acids, particularly oligonucleotides, for therapeutic applications has been described, e.g., in Dias and Stein, Mol. Cancer Ther. 1:347-55 (2002), Goodchild, Curr. Opin. Mol. Ther. 6:120-28 (2004), Kurreck, Eur. J. Biochem. 270:1628-44 (2003), Marcusson et al., Mol. Biotechnol. 12:1-11 (1999), Opalinska and Gewirtz, Nat. Rev. Drug Discov. 1:503-14 (2002), Ravichandran et al., Oligonucleotides 14:49-64 (2004), and Shi and Hoekstra, J. Control. Release 97:189-209 (2004). Therapeutic siRNAs have been described, e.g., in U.S. Pat. No. 7,989,612. Oligonucleotide decoys have been described, e.g., in US Application 20110166212.

In other embodiments, the therapeutic agent is a polypeptide (e.g., a protein). The polypeptide can be, e.g., a vaccine, an antibody, a transcription factor (e.g., a transcription factor responsive to extracellular signaling events, such as a Notch receptor intracellular domain fragment), a cytoplasmic protein (e.g., involved in signal transduction, such as a kinase or adaptor protein that functions by binding to phosphorylated protein epitopes), or a dominant-negative protein mutant (e.g., that interferes with normal signal transduction). The polypeptide can also be, e.g., a growth factor or protein hormone.

In still other embodiments, the therapeutic agent is a chemical compound. The chemical compound can be, for example, an antibiotic; antiviral agent; analgesic or combination of analgesics; anorexic; antihelminthic; antiarthritic; antiasthmatic agent; anticonvulsant; antidepressant; antidiabetic agent; antidiarrheal; antihistamine; antiinflammatory agent; antimigraine preparation; antinauseant; antineoplastic; antiparkinsonism drug; antipruritic; antipsychotic; antipyretic; antispasmodic; anticholinergic; sympathomimetic; xanthine derivative; cardiovascular preparation, such as a potassium or calcium channel blocker, beta-blocker, alpha-blocker, or antiarrhythmic; antihypertensive; diuretic or antidiuretic; vasodilator, including general, coronary, peripheral or cerebral; central nervous system stimulant; vasoconstrictor; cough and/or cold preparation, including a decongestant; hormone, such as estradiol or other steroid, including a corticosteroid; hypnotic; immunosuppressive; muscle relaxant; parasympatholytic; psychostimulant; sedative; or tranquilizer. By the methods of the present invention, drugs in all forms, e.g., ionized, nonionized, free base, acid addition salt, and the like may be delivered, as can drugs of either high or low molecular weight.

Diagnostic agents suitable for inclusion in the compositions of the invention include any nucleic acid, polypeptide or chemical compound useful for diagnostic methods, including, for example, fluorescent, radioactive, or radio-opaque dye. After compositions (e.g., pharmaceutical compositions) comprising an amphiphilic cationic polymer of the invention combined with a diagnostic agent have been administered to an organism, the polymer and/or diagnostic agent can be tracked using well-known techniques such as PET, MRI, CT, SPECT, etc.

Amphiphilic cationic polymers of the invention, when combined with therapeutic and/or diagnostic agents, will preferable form homogeneous complexes having a desirable size. For example, compositions of the invention can comprise amphiphilic cationic polymers complexed with a therapeutic agent (e.g., a nucleic acid), wherein the complexes have an average diameter of about 500 nm or less. Preferably, the complexes in the composition will be homogeneous and have an average diameter of about 50 nm to about 300 nm, about 100 nm to about 275 nm, about 150 nm to about 250 nm, or about 200 nm. As used herein, the term “homogenous,” when used to refer to polymer-therapeutic agent or polymer-diagnostic agent complexes, means that at least half of the complexes have a diameter that is the same as or within +/−20% of the average diameter of the complexes in the composition.

Compositions of the invention can further comprise an agent that enhances endosomal release. For example, lytic peptides may be included in the compositions. A “lytic peptide” is a peptide which functions alone or in conjunction with another compound to penetrate the membrane of a cellular compartment, particularly a lysosomal or endosomal compartment, to allow the escape of the contents of that compartment to another cellular compartment, such as the cytoplasm and/or nuclear compartment. Examples of lytic peptides include toxins, such as Diptheria toxin or Pseudomonas exotoxin.

Alternatively, compositions of the invention can further comprise an agent that facilitates the targeting of specific cell types. For example, the compositions can comprise an antibody or other agent that specifically binds to certain cell types.

Compositions of the invention, as described herein, find use in the manufacture of medicaments. Likewise, the compositions find use in methods of treating a condition. The medicament can be useful for treating the condition, and the condition can be a condition susceptible to treatment using a therapeutic agent found in the composition, e.g., as described further below.

Methods Utilizing Compositions of the Invention

The invention further provides methods of facilitating delivery of a therapeutic and/or diagnostic agent into a cell. The methods comprise contacting a cell with a composition of the invention and allowing a therapeutic and/or diagnostic agent contained in the composition to enter the cell. The cell can be located in vitro, e.g., as part of a primary culture of cells or part of a cell line, e.g., a CHO, C2C12, H4IIE or HSK (human skeletal muscle) cell line. Alternatively, the cell can be located in vivo, e.g., inside of an organism such as a human. The cell can be undifferentiated (e.g., a stem cell or progenitor cell) or at different stage of differentiation (e.g., a muscle cell, a liver cell, an endothelial cell, a blood cell, an intestinal mucosal cell, a nasal mucosal cell, a neuron, etc.).

Contacting a cell located in vitro can simply involve injecting the composition into the surrounding cell culture medium. Alternatively, the composition can be laid upon the cells after the cell culture medium has been removed. Contacting a cell located in vivo typically involves administering the composition to an organism such that the composition is able to contact the cell (e.g., a target cell). The administration of the composition to an organism can be performed by injection (e.g., intravenous injection, intramuscular injection, intraperitoneal injection, injection into the CNS, etc.). Preferably, the injection takes place in a location proximal to a target cell. For example, muscle cells can be targeted by intra-muscular injection, while liver cells can be targeted by intravenous injection. Alternatively, the composition can be administered to an organism by topical application (e.g., direct application to a tissue or open wound), by oral ingestion, or nasal application. The appropriate mode of administration will depend upon the target cells and the therapeutic or diagnostic agent present in the composition. Persons skilled in the art will readily be able to determine an appropriate route of administration for specific compositions of the invention.

Compositions of the invention can be administered to any of a variety of organisms, including microorganisms (e.g., bacteria, yeast), fungi, plants, and animals (e.g., birds, reptiles, marine animals, domesticated animals, pets, wild animals), particularly mammals. Examples of bacteria and yeast include bacteria and yeast that reside within or infect animals, such as E. coli, Salmonella, Mycobacteria, and the like. Examples of domesticated animals and/or pets include dogs, cats, mice, rats, guinea pigs, rabbits, pigs, cows, sheep, goats, horses, etc. Examples of wild animals include monkeys, apes, bears, lions, tigers, wolves, buffalo, deer, elk, moose, foxes, etc. Examples of birds include chicken and ducks. Preferably, the compositions of the invention are administered to a mammal, such as a mouse or a human.

The invention also provides methods of treating a condition in an organism by administering a composition of the invention, wherein the composition comprises a therapeutic agent suitable for treating the condition. The organism can be any organism described herein. Preferably the organism is a mouse or a human. The condition can be a genetic disease (e.g., an heritable disease or a congenital disease), an infection (e.g., a bacterial, fungal, viral, or other type of infection), a cardiovascular disorder (e.g., atherosclerosis, hypertension, etc.), a pulmonary disease (e.g., cystic fibrosis), a metabolic disease (e.g., diabetes type II), cancer, an immunological (e.g., autoimmune) disease, a neurological condition or disorder (e.g., pain, such as post-operative pain), etc. In particular embodiments, the disease is a muscular disease, such as muscular dystrophy (e.g., Duchenne's muscular dystrophy).

As used herein, the singular forms “a,” “an,” and “the” include plural referents unless the context clearly dictates otherwise. Thus, for example, reference to a polymer containing “an organic cation” includes reference to one or more of such organic cations.

The following examples will enable those skilled in the art to more clearly understand how to practice the present invention. It is to be understood that, while the invention has been described in conjunction with the preferred specific embodiments thereof, that which follows is intended to illustrate and not limit the scope of the invention. Other aspects of the invention will be apparent to those skilled in the art to which the invention pertains.

### EXAMPLES

### Example 1

### Synthesis of Exemplary Polycarbamate (PCM) Amphiphilic Cationic Polymers

Pluronic®-PEI polymers were synthesized according to the methods of Cho et al., Macromolecular Research, 14:348-353 (2006). Briefly, Pluronics were dried overnight in vacuo at 40° C. prior to modification, then activated with an excess of 1,1′-carbonyldiimidazole (CDI) in 10 ml of anhydrous acetonitrile. After stirring for 3 hours at room temperature, the reaction mixture was treated with 0.5 ml water for 20 minutes to neutralize the nonreacted CDI. An excess of PEI in 20 ml of ethanol was then mixed with the activated Pluronics and the mixture was stirred overnight. Next, the mixture was diluted with water and dialyzed against 20% aqueous ethanol for 24 hrs using a membrane tube (2000 Da molecular weight cutoff) to remove small molecular mass reagents, including PEI. The conjugates were further separated using cation exchange chromatography for the separation of unconjugated Pluronic from the conjugated form. The purified conjugates were dialyzed against water and lyophilized to obtain the final product. Synthesized polymers were characterized by Nuclear Magnetic Resonance (¹H-NMR) and elemental microanalysis for composition and molecular weight.

A partial list of PCM polymers of the invention that have been synthesized and tested include:

| | | | | Con- | |
|---|---|---|---|---|---|
| | Mw of reactants | | | jugated | Yield of |
| Data | Pluronic/PEG | | | percent of | copolymer |
| Code | Mw(Da)^{a} | HLB^{b} | PEI | PEI (%)^{c} | (%)^{d} |
| PCM-01 | L64 (2900) | 12-18 | 800 | 92.3 | 84.1 |
| PCM-02 | P85 (4600) | 12-18 | 800 | 88.4 | 79.2 |
| PCM-03 | F127(12600) | 18-23 | 800 | 79.7 | 74.5 |
| PCM-04 | L64 (2900) | 12-18 | 1,200 | 86.7 | 81.3 |
| PCM-05 | P85 (4600) | 12-18 | 1,200 | 85.4 | 77.8 |
| PCM-06 | F127(12600) | 18-23 | 1,200 | 81.2 | 79.3 |
| PCM-07 | L35 (1900) | 18-23 | 800 | 90.8 | 71.4 |
| PCM-08 | L44 (2200) | 12-18 | 800 | 87.9 | 82.7 |
| PCM-09 | L35 (1900) | 18-23 | 1,200 | 84.7 | 80.5 |
| PCM-10 | L44 (2200) | 12-18 | 1,200 | 82.5 | 78.5 |
| PCM-11 | P123 (5750) | 7-9 | 800 | 77.5 | 82.5 |
| PCM-12 | P123 (5750) | 7-9 | 1,200 | 80.2 | 78.4 |
| PCM-13 | PEG-6000^{e} | hydrophilic | 800 | 95.4 | 78.8 |
| PCM-14 | PEG-6000^{e} | hydrophilic | 1,200 | 92.3 | 75.9 |

^{a} & ^{b}Values for the average molecular weight (Mw) and the hydrophilic-lipophilic balance (HLB) were obtained from the manufacturer (BASF);
^{c}Values for conjugated % of PEI were determined using NMR and elemental microanalysis;
^{d}Yields were determined from the pluronic feed amount, assuming both ends were modified by PEI;
^{e}Polymers of PEG-6000 conjugated to PEI were synthesized for comparison with the amphiphilic cationic polymers of the invention.

Additional polymers of the invention comprising small organic amines (e.g., bis-aminopropyl piperazine (BAPP)) linked to either Pluronics (e.g., PluronicL64, PluronicP85) or Tween (e.g., Tween-20 (T20)) have been synthesized and tested, including the following:

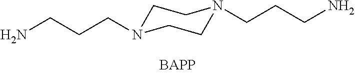 [SMILES: NCCCN1CCN(CCCN)CC1]

| Compd. | Description |
|---|---|
| 021 | L64-BAPP |
| 025 | P85-BAPP |
| 044 | T20-BAPP |

### Example 2

### Synthesis of Exemplary Dendron-Capped and Arginine-Capped Amphiphiles

For the synthesis of dendron-capped Pluronic® P85 amphiphilic polymers, Pluronic® P85 was activated with 1,1′-carbonyldiimidizole (CDI) and then mixed with an excess of ethylenediamine in 20% ethanol. After stirring overnight, the reaction mixture was diluted with distilled water and dialyzed for 24 hours against 20% ethanol using membrane tubes having a molecular weight cut-off of 2000 Da. The product was then lyophilized to obtain the intermediate NH2-P85-NH2 (P85-G0). The ¹H NMR (D₂O) spectrum for the P85-G0 was: δ PPO [—OCH₂CHCH₃)—, m] 1.14; δ PPO+PEO [—OCH₂CH(CH₃)—, —CH₂CH₂O—, m] 3.40-3.65; δ [—OCONHCH₂CH₂NH₂, m] 2.75-2.90.

Synthesis of P85-G0.5.

Next, P85-G0 was dissolved in methanol and added drop-wise to 100 equivalents of methyl acrylate maintained at room temperature. After 48 hours, methanol and unreacted methyl acrylate were removed under vacuum. The residue was precipitated with an excess of cold ethyl ether and dried under vacuum to remove ethyl ether, leaving a white solid, P85 G0.5. The ¹H NMR (MeOD) spectrum for the P85-G0.5 was: δ PPO [—OCH₂CHCH₃)—, m] 1.14; δ PPO+PEO [—OCH₂CH(CH₃)—, —CH₂CH₂O—, m] 3.40-3.65; δ [—OCONHCH₂CH₂NH₂, m] 2.75-2.92; δ PAMAM [—COOCH₃, m] 3.66; δ PAMAM [—CH₂COOCH₃, m] 2.51.

Synthesis of P85-G1.0.

P85-G0.5 was dissolved in methanol and added drop-wise to 100 equivalents of ethylenediamine kept at room temperature. After 48 hours, methanol and ethylenediamine were removed under vacuum. The residue was precipitated with an excess of ethyl ether to remove residual ethylenediamine and dried under vacuum to remove ethyl ether, leaving a pale yellow solid, P85-G1.0. The ¹H NMR (D₂O) spectrum for the P85-G1.0 was: δ PPO [—OCH₂CHCH₃)—, m] 1.14; δ PPO+PEO [—OCH₂CH(CH₃)—, —CH₂CH₂O—, m] 3.40-3.70; δ PAMAM [—CH₂CONH—] 2.45; δ PAMAM [—CONHCH₂—] 3.35; δ PAMAM [—CH₂CH₂—, m] 2.75-2.95.

Through iterative multistep reactions comprising Michael addition of methyl acrylate followed by amidation of ethylenediamine, as shown in Scheme-3, a series of dendron-capped Pluronic® P85 amphiphilic polymers was prepared.

The synthesis of arginine-modified amphiphile was performed according to the method of Kim et al., Biomaterials 30:658-664 (2009). Dendron-modified poloxamer (P85-G3) was reacted with excess of each of 1-Hydroxybenzotriazole (HOBT), 0-(Benzotriazol-1-yl)-N,N,N′,N′-tetramethyluronium hexafluorophosphate (HBTU), and Fmoc-Arg(pbf)-0H, and 8 equivalents of Diisopropylethylamine (DIPEA), keeping the reaction in anhydrous DMF for 1 day at room temperature. The reaction product was precipitated three times with an excess of diethyl ether, then mixed with an equal volume of piperidine (30% in DMF) at room temperature for 20 minutes, to remove the Fmoc groups of the coupled Fmoc-Arg(pbf)-0H. The reaction mixture was then precipitated again with diethyl ether and incubated with trifluoroacetic acid/triisopropylsilane/water (95:2.5:2.5 v/v/v) at room temperature for six hours to deprotect the pbf groups of coupled arginine residues. The final product (P85-G3-R) was dialyzed against ultrapure water overnight and lyophilized before use for analysis and assay. The ¹H NMR (D₂O) spectrum for the P85-G3-R was: PPO [—OCH₂CH(CH₃)—, m] 1.15; δ arginine [—HCCH₂CH₂CH₂CH₂NH—] 1.67; δ arginine [—HCCH₂CH₂CH₂NH—] 1.85; δ PPO+PEO [—OCH₂CH(CH₃)—, —CH₂CH₂O—, m] 3.40-3.78; δ PAMAM [—CH₂CONH—] 2.49; δ PAMAM [—CONHCH₂— and —CONHCH₂CH₂NHCO—] 3.37; δ PAMAM [—CH₂CH₂—, m] 2.76-2.98; δ arginine [—HCCH₂CH₂CH₂NH—] 3.25; δ arginine [—HCCH₂CH₂CH₂NH—] 3.86.

### Example 3

### Analysis of Amphiphilic Cationic Polymers Complexed with Nucleic Acids

Polymer/DNA complexes were prepared fresh immediately before use by gently vortexing a mixture of DNA and a polymer solution at various polymer/DNA weight ratios. The complexes were incubated at room temperature for 30 minutes in a 24 microliter volume and loading dye was then added. Samples were loaded onto a 1% agarose gel with ethidium bromide (0.1 μg/ml) in tris-acetate (TAE) buffer (100V, 40 min), and the gel was analyzed on a UV illuminator.

Zeta Potential measurements of polymer/DNA complexes were performed at 25° C. using Zetaplu Zeta Potential Analyzer (Brookhaven Instrument Co.) equipped with a 15 mV solid-state laser operated at a wavelength of 635 nm. Effective hydrodynamic diameter was measured by photon correlation spectroscopy using the same instrument equipped with Multi Angle option. The size measurements were performed at 25° C. at the angle of 90°. Polymer/DNA complexes were prepared in 0.9% Sodium Chloride (AQUALITE@SYSTEM, Hospira, Inc., IL, USA).

The morphologies of the polymer/DNA complexes were analyzed using Transition Electron Microscopy (TEM; Philips CM-10). The samples were prepared using negative staining with 1% phosphotungstic acid. Briefly, one drop of polymer/DNA complex solution was placed on a formvar and carbon coated carbon grid (Electron Microscopy Sciences, Hatfield, Pa.) for 1 hour, and the grid was blotted dry. Samples were then stained for 3 minutes. The grids were blotted dry again. Samples were analyzed at 60 kV. Digital images were captured with a digital camera system from 4pi Analysis (Durham, N.C.).

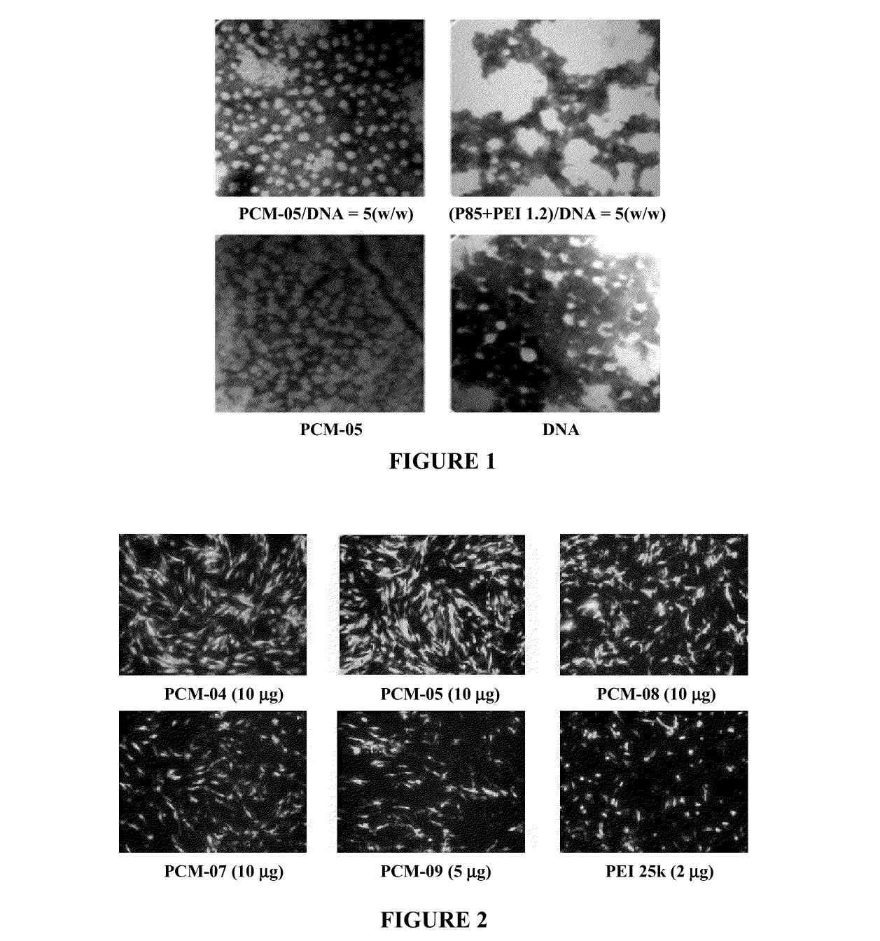

All of the PCM polymers condensed DNA into nano-sized particles at the polymer/DNA ratio of 2 and above, with highly homogenous hydrodynamic diameters around 200 nm. The particle size of PEI/DNA, in contrast, depended on the size of the PEI: PEI 0.8 k/DNA or PEI 1.2 k/DNA complexes formed aggregated particles >500 nm, whereas PEI 25 k formed very dense particle of around 100 nm. Physical mixtures of the same amount of Pluronic®, PEI and plasmid DNA produced aggregates with variable size ranging from 300 to 800 nm. The particle size of PCM polymers/DNA complex was further confirmed by TEM analysis, as shown in FIG. 1. Morphologically, these nanoparticles were well defined and uniformly distributed with sizes below 100 nm at a representative w/w ratio of 5. Physical mixtures of the same proportions of Pluronic®, PEI and plasmid DNA again showed aggregates of various size, characteristic of the interaction between free PEI and DNA reported previously. The clearly smaller particle size demonstrated by TEM in comparison with that from DLS analysis is most likely the results of TEM processing which required the samples to be dried, causing shrinkage in particle size.

### Example 4

### Amphiphilic Cationic Polymers have Low Cytotoxicity

Polymers of the invention were tested for their cytotoxicity to cells grown in culture. C2C12 myoblasts and Chinese Hamster Ovary (CHO) were grown in DMEM or RPMI-1640, respectively, and maintained at 37° C. and 10% CO₂ in a humidified incubator. 10⁴ cells per well were plated in a 96 well plate in 100 microliters of medium with 10% FBS (fetal bovine serum). After 24 hours, cell culture medium was replaced with serum-free medium and polymers were added at varying concentrations. PEIs were used as controls. Cytotoxicity was evaluated using the MTS assay by Cell Titer 96®Aqueous One Solution Proliferation Kit (Promega) 24 hours after the treatment with polymers.

The toxicity of PEI was clearly size-dependent, with higher molecular weight PEI showing higher toxicity. Cell viability dropped to <15% when treated with PEI 25 k at concentration of 10 μg/ml. Low molecular weight PEI (e.g., 0.8 k, 1.2K) showed very low cytotoxicity. All complexes showed remarkably lower cytotoxicity than that of PEI 25 k. In both cell lines, CHO and C2C12, toxicity of PCM-02, 03, 05, 06, 07, 08, 11, 12, 13, and 14 even at doses of 20 μg/ml was much lower than that with PEI 25 k at a dose of 5 μg/ml. This may be contributed to a more homogeneous particle size and a reduced density of the positively charged PEI. Toxicity was also associated with degree of hydrophobicity of Pluronics® within the PCMs, with higher toxicity observed for more hydrophobic Pluronics® such as PCM-01, 04, 09, 11 and 12 at high dose. This was further supported by the fact that hydrophilic PEG-PEI polymers (PCM-13 and 14) showed lower toxicity even at the highest concentration used.

### Example 5

### High Transfection Efficiency in C2C12 Cells Grown In Vitro

Amphiphilic cationic polymers of the invention were tested for their transfection efficiency. C2C12 myoblasts were grown as described in Example 4, above. 5×10⁴ cells per well were plated in a 24-well plate in 500 μl of medium with 10% FBS. After 24 hours, cell culture medium was replaced with serum-free medium and polymer/DNA complexes formulated with various ratios of polymer to DNA were added to the medium. 48 hours later, transfection efficiencies were determined quantitatively by flow cytometry (BD FACS calibur, BD). Relative efficiency was also recorded using an Olympus DP70 inverted microcopy.

FIG. 2 shows the GFP fluorescence of C2C12 cells following transfection with 1 μg of a GFP transgene complexed with 10 μg of PCM-04, 10 μg of PCM-05, 10 μg of PCM-07, 10 μg of PCM-08, or 5 μg of PCM-09. As a control, C2C12 cells were transfected with 1 μg of the GFP transgene complexed with 2 μg of PEI-25K. As shown in FIG. 2, the GFP fluorescence, and hence transfection efficiency, of C2C12 cells transfected with PCM-04, PCM-05, and PCM-08 is much higher than cells transfected with PEI 25 k.

### Example 6

### Synergistic Effects of Bonding Polyamines to Biocompatible Amphiphiles

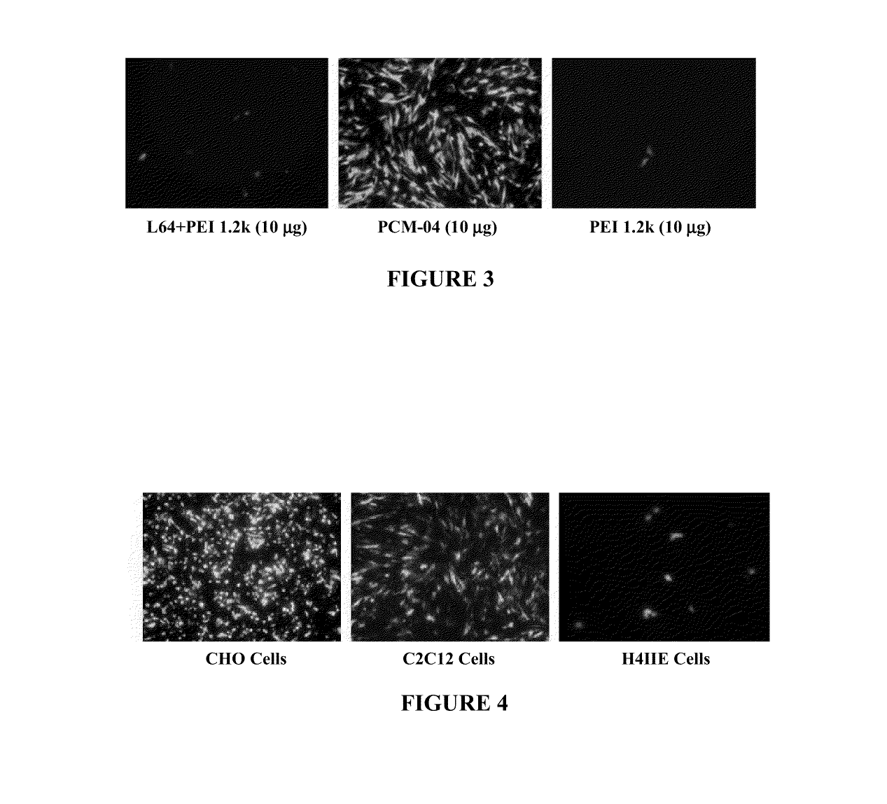

The transfection efficiency of 10 μg of PCM-04 complexed with 1 μg of a GFP transgene was compared to the transfection efficiency of (1) 10 μg of a mixture of Pluronic® L64 and PEI-1.2 k complexed with 1 μg of the GFP transgene, and (2) 10 μg of PEI-1.2 k complexed with 1 μg of the GFP transgene. C2C12 cells were grown, transfected, and analyzed as described in Example 5. FIG. 3 shows the GFP fluorescence of the C2C12 cells 48 hours post-transfection. As shown in FIG. 3, linking the polyamine PEI-1.2 k to the biocompatible amphiphile Pluronic® L64 dramatically increases the transfection efficiency as compared to simply mixing the two polymers together.

### Example 7

### Cell Line Dependent Transfection Efficiency

The transfection efficiency of PCM-04 for different cell lines was also tested. A GFP transgene was complexed with PCM-04 at a ratio of 5:1 (w/w) for transfection of C2C12 and CHO cells, and PCM-04 at a ratio of 10:1 (w/w) for transfection of rat hepatoma H4IIE cells. C2C12 and CHO cells were grown and transfected as described in Example 4. H4IIE cells were grown in DMEM with 10% FBS and transfected with same procedure as for C2C12 cells. The transfection efficiency was measured by GFP fluorescence of the transfected cells. As shown in FIG. 4, PCM-04 induced the highest transfection efficiency with CHO cells, an intermediate transfection efficiency with C2C12 cells, and a relatively low transfection efficiency with H4IIE cells.

### Example 8

### Enhanced Antisense Oligonucleotide-Mediated Exon Skipping in C2C12 E50 Cells Treated with Amphiphilic Cationic Polymers of Intermediate Size and HLB

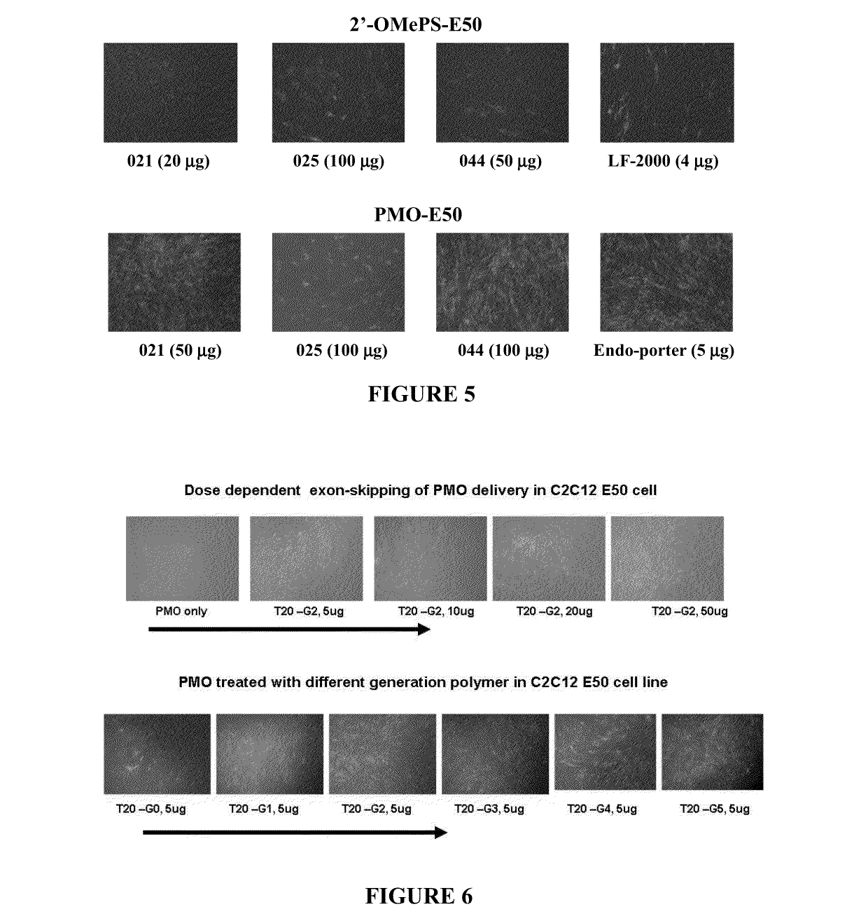

The polymers of the invention were also tested for their ability to transfect cells with antisense oligonucleotides. BAPP-based polymers PCM-021 (20 μg), PCM-025 (100 μg), and PCM-044 (50 μg) were complexed with 2 μg 2′-O-methyl phosphorothioate (2′-OMePS)-E50 antisense oligonucleotides. In addition, PCM-021 (50 μg), PCM-025 (100 μg), and PCM-044 (100 μg) were complexed with 5 μg PMO-E50 antisense oligonucleotides. The complexes were then transfected into C2C12 E50 cells. Antisense oligonucleotide-mediated skipping of exon 50 of the dysrophin gene in C2C12 E50 cells restores the reading frame of a GFP transgene, thus resulting in the expression of GFP protein. The results are shown in FIG. 5. For 2′-OMePS delivery, 4 μg Lipofectamine-2000 (LF-2000) complexed with 2 μg of 2′-OMePS-E50 was used as the control. For PMO delivery, 5 μg of Endo-porter complexed with 5 μg of PMO-E50 was used as the control. The transfection efficiency of 2′-OMePS-E50 using PCM-025 was comparable to that obtained with LF-2000, while the transfection efficiency using PCM-021 and PCM-044 was comparatively lower. Conversely, the transfection efficiency of PMO-E50 using PCM-021 and PCM-044 was comparable to that obtained using Endo-porter, while the transfection efficiency using PCM-025 was comparatively lower.

### Example 9

### PMO Antisense Oligonucleotide-Mediated Exon Skipping in C2C12 E50 Cells Using Different Doses and Generations of Tween-20 Dendrimers

Tween-20 (T20) dendrimers of the invention were tested for their ability to transfect C2C12 E50 cells with PMO and thereby stimulate skipping of Exon 50. Different doses (0 μg, 5 μg, 10 μg, 20 μg, or 50 μg) of T20 dendrimer generation 2 (T20-G2) were complexed with 5 μg of PMO and the resulting compositions administered to C2C12 E50 cells. T20-G2 dendrimer exhibited a significant dose-dependent increase in PMO delivery efficiency and exon skipping as compared to PMO alone at all doses tested, with GFP expression increasing in a dose-dependent manner until reaching a plateau around 10 μg T20-G2. See FIG. 6. PMO delivery efficiency was also improved with increased generation of T20 dendrimers, from G0 to G2. The GFP expression showed that at a dose of 5 μg, T20-G2 achieved high expression, with no obvious increase with higher generations. These results demonstrated that dendrimer size and optimum dose are key factors for antisense oligomer delivery.

### Example 10

### Enhanced Delivery in Muscle Cells In Vivo

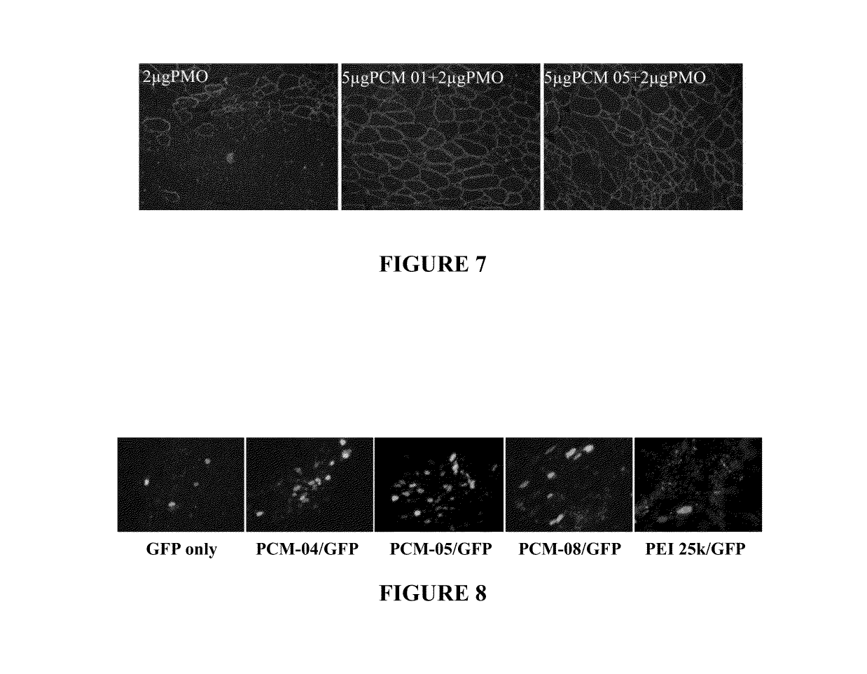

To test the in vivo transfection efficiency of the polymers of the invention, 2 μg of PMO-E23 antisense oligonucleotides were injected into the tibialis anterior (TA) muscles of mdx mice aged 4-6 weeks. The E23 antisense oligonucleotides were injected alone or complexed with 5 μg of either PCM-01 or PCM-05. Two weeks post-injection, the TA muscles were dissected out, sectioned, and stained for dystrophin protein. The number of dystrophin positive muscle fibers indicates the efficiency of the PMO transfection. Dystrophin protein appears as red, membrane-localized staining, as shown in FIG. 7. Over 50% of TA muscle fibers treated with PMO-E23 complexed with either PCM-01 or PCM-05 displayed increased dystrophin expression. In comparison, only 12-13% of muscle fibers treated with PMO-E23 alone expressed dystrophin.

Based on transfection efficiency and cytotoxicity in the cell culture systems, PCM-04, PCM-05 and PCM-08 polymers were selected for further examination of their potential for gene delivery in muscle by intramuscular injection. 10 μg of a GFP expression vector alone or complexed with 10 μg PCM-04, PCM-05, or PCM-08 was injected into the TA muscles of the mdx mice age 4-6 weeks, and GFP expression was examined 5 days post-injection. The results are shown in FIG. 8. The number of GFP-expressing muscle fibers was 75±11, 137±15 and 93±13 for PCM-04, PCM-05 and PCM-08, respectively. As a control, 10 μg of the GFP expression vector complexed with 5 μg PEI 25 k induced only 15-20 positive muscle fibers. Histologically, there was no clearly observable muscle damage in the muscles treated with the three PCMs at the dose used when compared to the muscles injected with saline only. In contrast, 5 μg PEI 25 k induced significant muscle damage with large areas of necrotic fibers and focal infiltrations.

The above examples are illustrative only and do not define the invention; other variants will be readily apparent to those of ordinary skill in the art. The scope of the invention is encompassed by the claims of any patent(s) issuing herefrom. The scope of the invention should, therefore, be determined not with reference to the above description, but instead should be determined with reference to the issued claims along with their full scope of equivalents. All publications, references, accession numbers, and patent documents cited in this application are incorporated by reference in their entirety for all purposes to the same extent as if each individual publication or patent document were so individually denoted.

What is claimed:

1. A composition comprising a therapeutic agent in combination with an amphiphilic cationic polymer, wherein the amphiphilic cationic polymer comprises a biocompatible amphiphile linked to an organic cation, and wherein the biocompatible amphiphile and the organic cation are linked by a biodegradable linker.

2. The composition of claim 1, wherein the amphiphilic cationic polymer has a structure selected from the group consisting of:
OC-LN-H-L-LN-OC (i);
OC-LN-L-H-L-LN-OC (ii); and
OC-LN-H-L-H-LN-OC (iii),
wherein H is a hydrophilic segment, L is a lipophilic segment, LN is a biodegradable linker, OC is an organic cation, and the dashes are covalent chemical bonds, and wherein the hydrophilic and lipophilic segments together constitute the biocompatible amphiphile.

3. The composition of claim 1, wherein the biocompatible amphiphile is an amphiphilic block copolymer.

4. The composition of claim 3, wherein the amphiphilic block copolymer has a structure selected from the group consisting of:
H[OCH₂CH₂]ₓ[OCH(CH₃)CH₂]_{y}OH (I);
H[OCH₂CH₂]ₓ[OCH(CH₃)CH₂]_{y}[OCH₂CH₂]_{z}OH (II);
H[OCH(CH3)CH₂]ₓ[OCH₂CH₂]_{y}[OCH(CH₃)CH₂]_{z}OH (III); 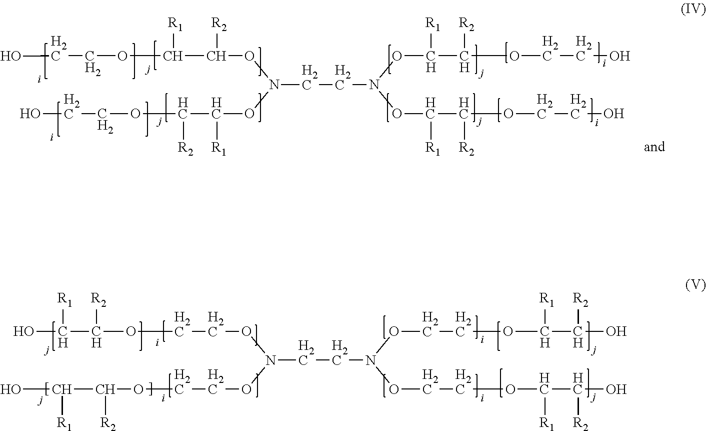
wherein x, y, z in formulas I-III each have a value from about 5 to about 80, and wherein i and j in formulas IV-V each have a value from about 2 to about 25.

5. The composition of claim 1, wherein the biocompatible amphiphile has a hydrophilic-lipophilic balance (HLB) of about 10 to about 26.

6. The composition of claim 1, wherein the biocompatible amphiphile has a size of about 1000 Da to about 10000 Da.

7. The composition of claim 1, wherein the organic cation is an amine.

8. The composition of claim 7, wherein the amine is selected from the group consisting of polyethylenimine (MW≦2000 Da), dendrimer (MW≦3000 Da), bis-aminopropyl piperazine (BAPP), and arginine.

9. The composition of claim 1, wherein the biodegradable linker is selected from the group consisting of an esteramine and a carbamate.

10. The composition of claim 1, wherein the therapeutic agent is a nucleic acid.

11. The composition of claim 10, wherein the nucleic acid is an oligonucleotide.

12. The composition of claim 1, wherein the composition forms a homogenous collection of particles having a diameter of about 50 nm to about 300 nm.

13. A pharmaceutical composition comprising a composition of claim 1 and a pharmaceutically acceptable carrier.

14. A method of facilitating delivery of a therapeutic agent into a cell comprising contacting the cell with a composition of claim 1 and allowing the therapeutic agent to enter the cell.

15. The method of claim 14, wherein the cell is contacted in vitro.

16. The method of claim 14, wherein the cell is contacted in vivo.

17. The method of claim 16, wherein the contacting step comprises applying the composition directly to an organism or injecting the composition into the organism.

18. The method of claim 14, wherein the cell is a muscle cell, a liver cell, an endothelial cell, a blood cell, a neuron, an intestinal mucosal cell, or a nasal mucosal cell.

19. A method of treating a condition in an organism comprising administering a composition of claim 1 to the organism, wherein the therapeutic agent is suitable for treating the organism's condition.

20. The method of claim 19, wherein the organism is a human.

21. The method of claim 19, wherein the condition is a muscular dystrophy.
